(19) United States
(12) Patent Application Publication
(10) Pub. No.: US 2024/0197618 A1
(43) Pub. Date: Jun. 20, 2024

(54) ROSEWOOD ESSENTIAL OIL NANOCAPSULES AND THEIR NANOENCAPSULATION PROCESS

(71) Applicant: Instituto Nacional de Pesquisas da Amazônia (INPA), Manaus (BR)

(72) Inventors: IVONE KETURA SILVA CABRAL, MANAUS (BR); DANIELLE MONTEIRO DE OLIVEIRA, MANAUS (BR); PAULO DE TARSO BARBOSA SAMPAIO, MANAUS (BR); NEWTON PAULO DE SOUZA FALCÃO, MANAUS (BR)

(73) Assignee: Instituto Nacional de Pesquisas da Amazônia (INPA), Manaus (BR)

(21) Appl. No.: 18/390,119

(22) Filed: Dec. 20, 2023

(30) Foreign Application Priority Data
Dec. 20, 2022 (BR) BR 10 20220260656

(51) Int. Cl. A61K 8/9789; A61K 8/11

(52) U.S. Cl. CPC A61K 8/9789; A61K 8/11

(57) ABSTRACT

The present invention is related to the field of biotechnology, more specifically to the area of natural products, such as nanocapsules of rosewood essential oil (Aniba rosaeodora Ducke) and its nanoencapsulation process. The use of rosewood essential oil (Aniba rosaeodora Ducke) is justified because of its linalool component. However, this raw material undergoes oxidative processes that interfere with its integrity, leading to the degradation of its volatile components and compromising the export process. In view of this context, the present invention discloses nanocapsules of rosewood essential oil and their encapsulation method, using the Nano spray dryer equipment, for faster drying, with higher quality and with the aim of promoting greater conservation of the product, in order to stimulate sustainable management, in the formation of new products based on Amazonian forest species.

### FIELD OF APPLICATION

The present invention is related to the field of biotechnology, more specifically to the area of natural products, such as nanocapsules of rosewood essential oil (Aniba rosaeodora Ducke) and its nanoencapsulation process. The nanocapsules were produced using nanoencapsulation and drying techniques to solve problems caused by the presence of light, heat, humidity and microorganisms, which degrade the essential oil.

### FUNDAMENTALS OF INVENTION

The chemical, cosmetic and perfumery industrial markets make use of resources and inputs found in plant species, due to the nobility of the chemical constituents. Among the chemical constituents appreciated by the perfume industry is linalool, which is used as a fixative and flavoring agent in perfumes. However, this noble content is only found in a few different species, such as rosewood (Aniba rosaeodora Ducke), for example.

Rosewood is a species native to the Amazon that produces essential oil with high levels of linalool. However, this raw material undergoes various chemical, physical and biological alterations (oxidative processes), which interfere with the integrity of the essential oil, leading to the degradation of its volatile components and compromising the export process, which causes a huge waste of the product, as well as all the labor applied from the process, the collection of botanical material, separation and extraction of the oil.

Today, industrial markets are already using emulsion, micro-emulsion and nano-emulsion formation techniques to encapsulate food, pharmaceutical and chemical products, which has revolutionized the fields of their application. However, state-of-the-art equipment such as the Nano spray dryer is not always used in these techniques, in order to improve the quality and refinement of these products.

Encapsulation is a method of protecting solids, liquids or gases from environmental conditions (humidity, light and air) by means of a natural or synthetic polymer layer. The benefits of encapsulation include increasing the shelf life of the volatile compound and controlling the chemical reaction between two active species. Industry is increasingly looking for biopolymers that can be used as encapsulating materials.

In view of the aforementioned problems, the present invention aims to provide a bioproduct through nanotechnology, which are nanocapsules of rosewood essential oil, produced using the Nano spray dryer equipment, for faster drying of the same, with higher quality and with the aim of promoting greater conservation of the product, against oxidative agents.

The fact that a natural biopolymer is used to form the wall of the nanocapsules contributes to the balance of the environment, because once the essential oil has been extracted from the capsule, it will dissolve and consequently will not generate polluting waste in the environment.

Nanocapsules are capable of preserving the physical and chemical characteristics of the essential oil for long periods of time, in order to stimulate sustainable management in the formation of new products based on forest species. This product could help optimize logistics for transporting rosewood essential oil, add value to the species and contribute to the Amazon's bioeconomy in terms of its non-timber resources.

### STATE OF THE ART

The scientific article “Nano spray drying for encapsulation of pharmaceuticals” refers to the process of encapsulating pharmaceuticals and liquid substances using the Nano Spray Drying technique and the key elements for this.

understanding, which plays an important role in converting formulations into powders liquid pharmaceutical for subsequent encapsulation. In addition, the article discusses the influence of process parameters on the final properties of the powders, such as particle size, morphology, encapsulation efficiency, drug loading and release.

Unlike the present invention, this document only discloses a review of scientific literature, aimed at showing the state of the art, the potential of the equipment Nano spray dryer and its better performance compared to other equipment, for the atomization and encapsulation of organic compounds in liquid form. The article does not disclose the use of this equipment for the nanoencapsulation process of rosewood essential oil (Aniba rosaeodora Ducke), which means that even if the same drying equipment (in this case the Nano spray dryer) is used in the article and in the present invention for the finalization of products, there is no guarantee that the end result will be the same and that the rosewood essential oil nanocapsule will be obtained.

The scientific article “Characterization Of Rosewood and Cinnamon Cassia Essential Oil Polymeric Capsules: Stability, Loading Efficiency, Release Rate And Antimicrobial Properties” reveals a process for encapsulating rosewood and cinnamon essential oils using Tween 80 and poly (butylene adipate-co-terephthalate) (PBAT) as a wall material. The capsules were obtained after evaporation of the solvent and evaluated by scanning electron microscopy (SEM), thermogravimetric analysis (TGA), loading efficiency (LE), release and antimicrobial tests.

However, the present invention differs from the scientific article in that it uses a non-toxic natural biopolymer, unlike poly (butylene adipate-co-terephthalate) (PBAT), which is a synthetic product derived from petroleum, which can easily interfere with the physicochemical properties of rosewood essential oil, as well as the stability of the nanocapsules. In addition, the present invention is made on a nanometric scale, which offers greater stability and encapsulation efficiency due to the size of the scale.

Another difference is that in the article, the encapsulation of the oil is done only under magnetic stirring and evaluated in morphological and thermogravimetric terms. In the present invention, the product was made under high homogenization pressure in a vortex at 1000 rpm and in a time interval of 5 min per sample. This process was clearly optimized compared to the one cited in the article, which took 12 hours under agitation.

The invention cited in the article also differs in that it was considered finished after homogenization, remaining in a liquid medium and on a micro scale. Which can easily be degraded by the external environment (light, humidity, temperature, micro-organisms) . The nanocapsule of the present invention was subjected to the drying process in the Nano spray Dryer, equipment which not only allows the encapsulated products to be reduced from the micro to the nano scale, but also guarantees the stability and high quality of the nanocapsules produced. Guaranteeing a new, highly refined product with high commercial value.

Chinese document CN106076217 describes a citral nanocapsule under acidic conditions and its preparation method. The nano capsule is prepared from gelatine, gum arabic, citral, Tween 20 and transglutaminase, where gelatine and gum arabic are wall materials and citral is the core, Tween 20 is an emulsifier and transglutaminase is a curing agent.

However, the Chinese document reveals a citral nanocapsule prepared using the coacervation technique, where it was possible to create the nanocapsules through magnetic stirring and temperature variation, and in addition, they were produced for use in drinks, medicines, toothpaste, chewing gum and cigarettes and especially acidic drinks. The nanocapsule of the present invention originated from high agitation combined with high vortex pressure, in addition to the main element being the essential oil of rosewood (Aniba rosaeodora Ducke) .

In addition, the Chinese document used a stronger sulphating agent than the one in the present invention, Tween 20, which has huge hydrophilic polyoxyethylene groups and is also used as a detergent, less suitable for preparing semi-solid and solid solutions. Another difference is the use of gelatine together with gum arabic for wall formation, which was not necessary in the present invention, since we used Tween 80, which guaranteed total stability of the nanocapsule walls.

Finally, the Chinese document did not use a Nano spray dryer to dry the nanocapsules, but instead used a high-temperature method and solvent evaporation.

The Brazilian document BR102020020385-1 describes an optimized process for obtaining a microencapsulated bioinsecticide based on Azadirachta indica A. Juss. (Meliaceae), formed by a polymeric system with gum arabic and maltodextrin as wall material with conditions for drying the bioproduct in a Spray dryer.

Unlike the present invention, the document discloses a microencapsulated bioinsecticide, with a difference in the size of its scales, where micro-sized products are still susceptible to the influence of the external environment and nano-scale products do not suffer this type of influence.

In addition, the bioinsecticide was developed with Azadirachta indica A. Juss. (Meliaceae), a shrub species, while the present invention was developed with Aniba rosaeodora Ducke (Rosewood), a forest species. Although the drying process in both documents was carried out in similar equipment (Spray Dryer), the way in which the drying was carried out differed in terms of drying time, feed time, flow rate, temperature, pressure and drying mode.

While the nanocapsule of the present invention was dried in an open system using water, the one in the Brazilian document was dried in a closed system using an organic solvent. The sizes of the nozzles were also different, since the scale of the present invention was nano and had greater refinement and quality than that of the Brazilian document.

The scientific article, “Minerals, Essential Oils, And Biological Properties Of Melissa Officinalis L” describes the mineral elements, chemical composition, antioxidant and antimicrobial activity of the Melissa officinalis plant. For this study, its essential oil was extracted by hydrodistillation of the dried leaves using a Clevenger-type dry leaf apparatus and analyzed using two techniques: gas chromatography coupled with flame ionization (GC-FID) and gas chromatography coupled with mass spectrometry (GC-MS) . Eighteen minerals were identified (As, Br, K, La, Na, Sb, Sm, Ba, Ca, Ce, Co, Cr, Cs, Fe, Rb, Sc, Th and Zn) and 78 compounds were identified in the essential oil, representing 94.090% of the total oil and yields of 0.470%. The essential oil showed high antimicrobial activity against microorganisms, mainly five human pathogenic bacteria, one yeast, Candida albicans and two phytopathogenic fungi.

However, the species used in the present invention was different (Aniba rosaeodora Ducke), and the rosewood samples were prepared for oil extraction by collecting, sorting, drying, grinding, weighing and extracting them.

For the extraction, a thermal blanket was also used in the exhaust pipes in order to conserve the entire cold and hot temperature of the Clevenger apparatus, which was not described in the scientific article. Another difference is that dry biomass was also extracted from branches and not just leaves, as described in the scientific article.

### SUMMARY OF THE INVENTION

The use of rosewood essential oil (Aniba rosaeodora Ducke) in the chemical, cosmetic and perfume industries is justified by its component linalool, which is used as a fixative and flavoring agent in perfumes. However, this raw material undergoes various chemical, physical and biological alterations (oxidative processes), which interfere with its integrity, leading to the degradation of its volatile components and compromising its export process.

In view of this context, the present invention discloses a bioproduct, which are nanocapsules of rosewood essential oil and their encapsulation method, using the Nano spray dryer equipment, to dry them faster, with higher quality and with the aim of promoting greater conservation of the product against oxidative agents, in order to stimulate sustainable management, in the formation of new products based on Amazonian forest species.

### BRIEF DESCRIPTION OF THE FIGURES

The invention can be better understood by briefly describing the following figures:

FIG. 1 shows a schematic representation of the process of extracting essential oil from the leaves and branches of Pau-rosa (Aniba rosaeodora Ducke): (1) collection of rosewood leaves and branches; (2) sorting; (3) drying in an oven at 45ºC; (4) grinding the samples; (5) weighing the dry biomass; (6) extraction of oil using a Clevenger apparatus; (7) crude rosewood oil.

FIG. 2 illustrates the schematic representation of the process of nanoencapsulation and drying of the essential oil from the leaves and twigs of Pau-rosa (Aniba rosaeodora Ducke): (a) homogenization of distilled water+synthetic or natural polymer and Pau-rosa essential oil for the formation of the nano emulsion; (b) drying of the nano emulsion in the Nano Spray Dryer for the formation of the Pau-Rosa nanocapsule.

### DETAILED DESCRIPTION OF THE INVENTION

The invention can be better understood through the following detailed description, in line with the figures.

The present invention describes a bioproduct, in the form of nanocapsules of the essential oil of rosewood (Aniba rosaeodora Ducke) and its method of nanoencapsulation, in order to promote the preservation of the product against oxidative agents.

The nanocapsules comprise at least one natural or synthetic polymer, crude rosewood (Aniba rosaeodora Ducke) essential oil, at least one emulsifier and distilled water. Optionally, the nanocapsule may additionally comprise at least one organic solvent and/or at least one long-chain triacylglycerol and/or at least one organic phase surfactant.

The natural or synthetic polymer has the function of forming the walls of the oil nanocapsules, i.e. encompassing and encapsulating the oil. The natural polymer being selected from the group comprising: commercial gum arabic (GA), cashew gum, cassava gum, xanthan gum, white rosin resin, yam gum, corn starch, rubber tree latex, milk and soy protein, cellulose, rice protein or guarana or a combination thereof; and the synthetic polymer being selected from the group comprising: poly (e-caprolactone) (PCL), alginate, maltodextrin, polystyrene, polyvinyl chloride, polypropylene or polyethylene terephthalate or a combination thereof. The natural or synthetic polymer has concentrations of 1 g/ml to 5 g/mL.

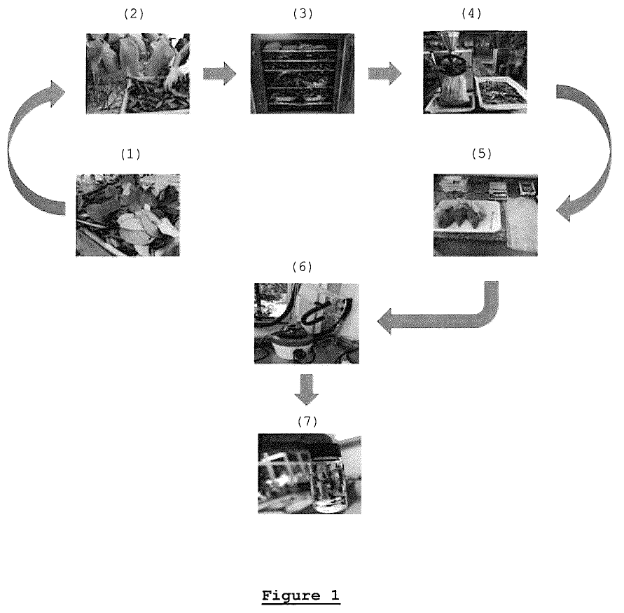

The crude essential oil of rosewood (Aniba rosaeodora Ducke) was extracted from the leaves and branches of different trees in the plantations of the National Institute for Amazonian Research (INPA), as shown in FIG. 1. First of all, rosewood leaves and branches are collected (1), then the collected leaves and branches are sorted (2) and dried in ovens at temperatures ranging from 40° C. to 65° C. for periods of 48 to 72 hours (3) . These samples are then ground using a knife mill, which cuts them up and turns them into powder (4), the dry biomass is weighed on a precision scale (5), and finally, the essential oil is extracted using the hydrodistillation method in the Clevenger apparatus (6), where the plant biomass is subjected to constant boiling in distilled water for 3 to 9 hours to extract all the essential oil (7) . Rosewood essential oil has a concentration in the nanocapsule of between 0.5% and 2.5% by volume, with a purity level of over 80% for the linalool present in rosewood essential oil.

The emulsifier has the function of providing greater stability, viscosity and homogeneity for the nanocapsules. The emulsifier was selected from the group comprising: polysorbate 80, polysorbate 20, carrageenan agar, glyceryl monoesterate, lauryl cetyl alcohol, alcohol, or a combination of these, with a concentration of 0.1 g/mL and 10 g/mL.

Distilled water solves all the solid components present in the solution, providing complete homogenization of the components to form the nanocapsule and its concentration varies from 100 mL to 300 mL in the total volume present in the solution.

The organic solvent has the function of dissolving all the solid compounds present in the solution, providing greater homogenization in the samples: acetone, hexane, xylene, benzene, methyl alcohol, ethyl alcohol, methylene chloride, ethyl acetate, ethanol, methyl ethyl ketone, toluene or dichloromethane, or a combination of these; the concentration varies from 0.1 g/mL to 10 g/mL, depending on the concentrations of essential oil and polymers applied. For every 0.5 ml of essential oil, you need 1 g of natural or synthetic polymer and 1.5 ml of organic solvent. As the concentrations of essential oil and natural or synthetic polymer increase, so do the concentrations of organic solvent, both proportionally.

The long-chain triacylglycerol has the function of providing greater stability, viscosity and homogeneity for the nanocapsules, and is selected from the group comprising: miritol, oleic acid, linoleic acid, stearic acid, butyric acid or a combination thereof; with a concentration of 0.1 g/mL to 10 g/mL.

The surfactant has the function of providing greater stability, viscosity and homogeneity for the nanocapsules, such as sorbitan monostearate, with a concentration of 0.1 g/mL to 10 g/mL.

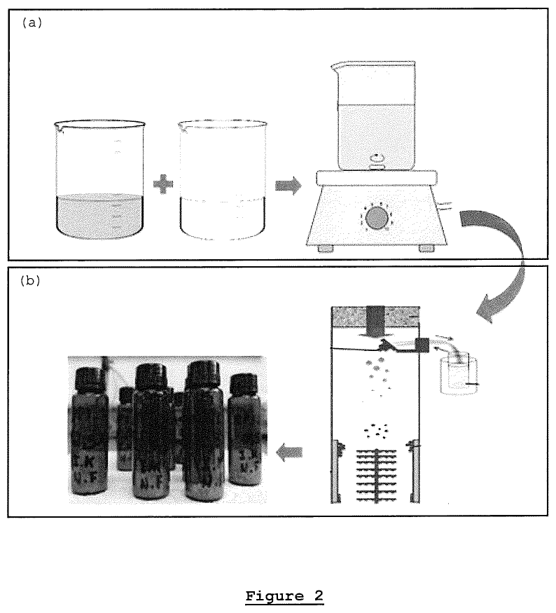

The process of nanoencapsulating crude rosewood essential oil (Aniba rosaeodora Ducke), as can be seen in FIG. 2, comprises the following steps:
(a) Homogenize at least one natural or synthetic polymer, the crude rosewood essential oil, at least one emulsifier and distilled water in a container to completely form the nano-emulsion;
(b) Dry the nano-emulsion to form the nanocapsules containing rosewood essential oil.

In step (a), 1 g/mL to 5 g/mL of natural or synthetic polymer, 0.5% to 2.5% by volume of crude rosewood essential oil, 0.1 g/mL and 10 g/mL of emulsifier and 100 mL to 300 mL of distilled water are added to a container and homogenized for 30 seconds to 15 min until a single phase is obtained. Optionally, when homogenization takes place using a vortex, the speed varies from 1000 rpm to 5000 rpm; when homogenization takes place using phase inversion, i.e. the oily or aqueous phase is heated in a water bath, and the components are heated and kept at a temperature of 70° C. to 75° C. until all the components are solubilized. The oily or aqueous phase is made up of the polymer, crude rosewood essential oil and distilled water. The organic phase is made up of the emulsifier, organic solvent, organic phase surfactant and distilled water.

Optionally, after step (a), the emulsion can be filtered to avoid any residues of the solid compounds used in the mixture. The homogenized solution is stirred for the complete formation of the nano-emulsion using a vortex, digital homogenizer or magnetic stirrer, with constant stirring from 1000 rpm to 5000 rpm for 30 seconds to 15 min, for subsequent drying in step (b). Filtration takes place using a glass funnel with 40 to 47 mm mesh paper filters for subsequent drying. The nano-emulsion has a single, homogeneous phase and can have a viscous or cloudy appearance.

In step (b), the nano-emulsion obtained undergoes the drying process. Drying is carried out in a Nano spray dryer, which has an injector nozzle with a diameter of between 0.5 and 1.5 mm. Drying of liquid components takes place by means of particle atomization, i.e. through high pressure and circulation of hot air inside the Nano spray dryer, fed by a peristaltic pump, with a temperature of 100° ° C. to 200° C. and a drying time of the nanocapsules equivalent to 1:30 h per 100 mL of solution and 2 h per 200 ml of solution. After this process, the samples are finished and ready to be collected, stored and analyzed.

### EXAMPLES

### Example 1: Preparation of Nanocapsules with Rosewood Essential Oil

Leaves and young branches of rosewood trees (Aniba rosaeodora Ducke) were collected. After collection, the samples were sorted and then subjected to the drying process. The material was dried in an industrial oven at a temperature of 45° C. for 48 hours. The dried biomass was then ground in a knife mill and weighed on a precision scale. 200 grams of the crushed material (rosewood leaves and twigs) were weighed separately and then added to a flask with 1 L of distilled water. The mixture underwent steam hydrodistillation in a Clevenger apparatus for a period of three hours for each sample. The extracted essential oil was stored in amber glass bottles with a stopper and placed in the fridge at −18° C. to fully preserve its volatile compounds.

The following were used to prepare the nanocapsules: commercial gum arabic in concentrations of 1%, 3% and 5% m/m; crude rosewood oil extracted from leaves and branches, in the following concentrations: 0.5%, 1.5% and 2.5% ml; the surfactants polysorbate 80, sorbitan monostearate and distilled water.

### Example 2: Preparation of Samples With Rosewood Essential Oil, Gum Arabic and Water

A total of 24 emulsion samples were made, 12 with essential oil extracted from leaves and 12 with essential oil extracted from twigs. In this treatment we used gum arabic (encapsulating biopolymer), rosewood essential oil and distilled water. Each sample was prepared in different proportions (for every 0.5 ml of oil, 1.0 g of gum was added, for emulsions of 1.5 ml of oil, 3.0 g of gum was added and for emulsions of 2.5 ml of oil, 5.0 g of gum was added) . After weighing the proportions of oil and gum, 100 ml of distilled water was added to all the samples. The solutions were mixed manually for 2.0 min until all the components were solubilized. After this process, each solution was placed in a vortex under constant stirring at 1000 rpm for 5.0 min for the complete formation of the nano-emulsions.

### Example 3: Sample Preparation with Oil, Gum Arabic, Water Plus Polysorbate 80 (Tween 80)

A total of 24 emulsion samples were made, 12 with oil extracted from leaves and 12 with oil extracted from twigs. In this treatment we used the surfactant polysorbate 80 in order to provide greater stability to the nanocapsules produced. The samples were prepared in the same proportions as the first treatment with the addition of 0.20 ml of polysorbate 80 to each formulation.

The solutions were mixed manually for 2.0 min until all the components were solubilized. After this process, each solution was placed in a vortex under constant stirring at 1000 rpm for 5.0 min for the complete formation of the nano-emulsions.

### Example 4: Sample Preparation With Oil, Gum Arabic, Water Plus Sorbitan Monostearate (SPAN 60)

This treatment involves the inversion of two phases: oily (aqueous) and organic. The same concentrations of gum arabic and rosewood oil as described above were used to prepare the solutions, plus the surfactant sorbitan monostearate and distilled water. Twenty-four emulsion samples were also prepared, 12 with oil extracted from leaves and 12 with oil extracted from twigs.

Preparation of the aqueous phase: gum arabic was solubilized in 50 mL of distilled water and oil was added, the solution was mixed manually for 2.0 min at room temperature.

Preparation of the organic phase: 0.20 g of sorbitan monostearate was placed in 50 mL of distilled water, then the solution was heated in the microwave for 2.0 min to completely dilute the surfactant.

The two phases were prepared simultaneously, then the organic phase was poured into the aqueous phase and stirred manually for 3.0 min. The solution was then homogenized using a vortex at a speed of 1000 rpm for 5.0 min in order to completely form the emulsions.

After this process, all the samples were placed in the refrigerator, where they were left to rest for 12 hours and then dried in the Nano Spray Dryer.

The nano-emulsions were then taken to the Thematic Laboratory of Microscopy and Nanotechnology for the drying process. Drying was carried out in the Nano spray dryer using the particle atomization method, where it was possible to control the feed, flow, temperature and drying time of the nanocapsules using small silicone hoses, in which one hose feeds the solution into the device and another waste hose discharges the waste produced from the solution out of the device. The drying time of the rosewood oil nanoencapsulates was 1.30 hours, at a temperature of 110° C. for each 100 mL solution. After this process, the samples were finished and ready to be collected and weighed.

The present invention has been disclosed in this descriptive report in terms of its preferred mode. However, other modifications and variations are possible from the present description, and are still within the scope of the invention disclosed herein.

### REFERENCE SIGNS

1—Collecting rosewood leaves and branches;
2—Screening;
3—Drying in an oven at 45° C.;
4—Grinding the samples;
5—Weighing the dry biomass;
6—Oil extraction using a Clevenger;
7—Crude rosewood oil;
a—Homogenization of distilled water+synthetic or natural polymer and rosewood essential oil to form the nano-emulsion;
b—Drying the nano-emulsion in the Nano Spray Dryer to form the Pau-Rosa nanocapsule.

1. Essential oil nanocapsule CHARACTERIZED by the fact that it comprises at least one natural or synthetic polymer, crude rosewood essential oil (Aniba rosaeodora Ducke), at least one emulsifier and distilled water.

2. Nanocapsule according to claim 1, CHARACTERIZED by the fact that it comprises at least one organic solvent and/or at least one long-chain triacylglycerol and/or at least one organic phase surfactant.

3. Nanocapsule according to claim 1, CHARACTERIZED in that the natural or synthetic polymer has a concentration of 1.0 g/mL to 5.0 g/mL and the natural polymer is selected from the group comprising: commercial gum arabic, cashew gum, cassava gumi, xanthan gum, white rosin resin, yam gum, corn starch, rubber tree latex, milk and soy protein, cellulose, rice protein, guarana or a combination thereof; and the synthetic polymer being selected from the group comprising: poly (e-caprolactone) (PCL), alginate, maltodextrin, polystyrene, polyvinyl chloride, vinyl acetate, polypropylene, polyethylene terephthalate or a combination thereof.

4. Nanocapsule, according to claim 1, CHARACTERIZED by the fact that the crude essential oil of rosewood (Aniba rosaeodora Ducke) has a concentration of 0.5% to 2.5% by volume and is extracted from leaves and branches.

5. Nanocapsule according to claim 1, CHARACTERIZED in that the emulsifier has a concentration of 0.1 g/mL and 10 g/mL and is selected from the group comprising: polysorbate 80, polysorbate 20, carrageenan agar, glyceryl monoesterate, lauryl alcohol, cetyl alcohol or a combination thereof.

6. Nanocapsule according to claim 1, CHARACTERIZED in that the distilled water has a concentration of 100 mL to 300 mL in the total volume of the nanocapsule.

7. Nanocapsule according to claim 2, CHARACTERIZED in that the organic solvent has a concentration of 0.1 g/mL to 10 g/mL and is selected from the group comprising: acetone, exane, xylene, benzene, methyl alcohol, ethyl alcohol, methylene chloride, ethyl acetate, ethanol, methyl ethyl ketone, toluene, dichloromethane or a combination thereof.

8. Nanocapsule according to claim 2, CHARACTERIZED in that the long-chain triacylglycerol has a concentration of 0.1 g/mL to 10 g/mL and is selected from the group comprising: miritol, oleic acid, linoleic acid, stearic acid, butyric acid or a combination thereof.

9. Nanocapsule according to claim 1, CHARACTERIZED in that the surfactant has a concentration of 0.1 g/mL to 10 g/mL and is sorbitan monostearate.

10. Nanoencapsulation process for crude rosewood essential oil (Aniba rosaeodora Ducke), CHARACTERIZED by the fact that it comprises the following steps:
(a) Homogenize at least one natural or synthetic polymer, the crude rosewood essential oil, at least one emulsifier and distilled water in a container to completely form the nano-emulsion;
(b) Dry the nano-emulsion to form the nanocapsules containing rosewood essential oil.

11. Process according to claim 10, CHARACTERIZED by the fact that in step (a) 1 g/mL to 5 g/mL of natural or synthetic polymer is added to a container, 0.5% to 2.5% by volume of crude rosewood essential oil, 0.1 g/mL and 10 g/mL of emulsifier and 100 mL to 300 mL of distilled water and homogenized for 30 seconds to 15 min.

12. Process, according to claim 10 or 11, CHARACTERIZED by the fact that still in step (a) when homogenization occurs by means of a vortex, the speed varies from 1000 rpm to 5000 rpm; when homogenization occurs by means of phase inversion, the oily (aqueous) phase being heated in a water bath, and the components are heated and maintained at a temperature of 70° C. to 75° C., until all the components are solubilized.

13. Process according to any one of claims 10 to 12, CHARACTERIZED by the fact that in step (a), after obtaining a single phase, the homogenized solution is stirred for the complete formation of the nano emulsion, by means of a vortex, digital homogenizer or magnetic stirrer, the stirring being constant from 1000 rpm to 5000 rpm for 30 seconds to 15 min.

14. Process according to any one of claims 10 to 13, CHARACTERIZED by the fact that still in step (a), optionally the homogenized solution is filtered.

15. Process according to claim 14, CHARACTERIZED by the fact that step (a) comprises, after the nano-emulsion has been formed, filtering it through a glass funnel with 40 to 47 mm mesh paper filters.

16. Process, according to claim 10, CHARACTERIZED by the fact that in step (b) the nano emulsion undergoes the drying process in a Nano spray dryer, which has an injector nozzle with a diameter ranging from 0.5 to 1,5 mm, with a temperature of 100° C. to 200 ° C. and a drying time of the nanocapsules equivalent to 1.30 hours per 100 ml of solution and 2 h per 200 ml of solution.
